(19) United States
(12) Patent Application Publication
(10) Pub. No.: US 2026/0260448 A1
(43) Pub. Date: Sep. 3, 2026

(54) X-RAY DIAGNOSTIC APPARATUS, MEDICAL IMAGE PROCESSING APPARATUS, AND CONTROL METHOD FOR X-RAY DIAGNOSTIC APPARATUS

(71) Applicant: CANON MEDICAL SYSTEMS CORPORATION, Otawara-shi (JP)

(72) Inventors: Masaki AKIYAMA, Nasushiobara (JP); Akihito TAKAHASHI, Nasushiobara (JP)

(73) Assignee: CANON MEDICAL SYSTEMS CORPORATION, Otawara-shi (JP)

(21) Appl. No.: 19/539,918

(22) Filed: Feb. 13, 2026

(30) Foreign Application Priority Data
Feb. 28, 2025 (JP) 2025-031538
Jan. 9, 2026 (JP) 2026-002874

(51) Int. Cl. G06V 10/25; A61B 6/00; A61B 6/06; A61B 6/12; A61B 6/40; A61B 34/20; G06V 20/60

(52) U.S. Cl. CPC G06V 10/25; A61B 6/06; A61B 6/12; A61B 6/4035; A61B 6/487; A61B 6/54; A61B 34/20; G06V 20/60; A61B 2034/2065; G06V 2201/034

(57) ABSTRACT

In one embodiment, an X-ray diagnostic apparatus includes an X-ray tube, an X-ray collimator, an X-ray detector, and processing circuitry. The X-ray tube generates X-rays. The X-ray collimator forms an opening having higher X-ray transmittance than other regions by using either or both of an X-ray filter configured to attenuate the X-rays and collimator blades configured to shield the X-rays. The X-ray detector detects the X-rays transmitted through an object. The processing circuitry recognizes at least one of a region of interest in the object and a medical device inserted into the object by image recognition on the basis of a detection signal from a detection region of the X-ray detector corresponding to a region of the opening. The processing circuitry outputs a result of the image recognition.

### CROSS-REFERENCE TO RELATED APPLICATION

This application is based upon and claims the benefit of priority from Japanese Patent Application No. 2025-031538, filed on February 28, 2025, and No. 2026-002874, filed on January 9, 2026, the entire contents of which are incorporated herein by reference.

### FIELD

Disclosed embodiments relate to an X-ray diagnostic apparatus, a medical image processing apparatus, and a control method for the X-ray diagnostic apparatus.

### BACKGROUND

In an X-ray diagnostic apparatus such as an X-ray angiography system, X-rays generated by an X-ray tube are emitted toward an object such as a patient, and an X-ray image of the object is acquired by using an X-ray detector to detect the X-rays that have passed through the object.

X-rays generated by the X-ray tube are directed toward the object after passing through, for example, a component referred to as an X-ray collimator (i.e., X-ray aperture unit). Inside the X-ray collimator, there is provided an aperture-defining X-ray collimator unit in which collimator blades configured to shield X-rays are arranged to define an aperture opening for transmitting the X-rays. Inside the X-ray collimator, an X-ray filter may also be provided as needed. The X-ray filter attenuates X-rays in regions other than a region of interest (ROI) and defines a filter opening through which X-rays are transmitted. The X-ray filter is also referred to as an ROI filter.

The X-ray collimator substantially blocks X-rays in regions other than the aperture opening, whereas the X-ray filter transmits X-rays in regions other than the filter opening at a predetermined level of attenuation.

A treatment method called interventional radiology (IVR) is widely performed by using an X-ray diagnostic apparatus such as an X-ray angiography system. IVR is translated into Japanese as “imaging-guided therapy”. In IVR, as the term suggests, the inside of the body is visualized for an interventional doctor using an X-ray diagnostic apparatus while a thin medical device is inserted into blood vessels to diagnose or treat a target disease. Aspects of medical devices to be inserted into blood vessels include a thin tube called a catheter, a balloon and/or stent that is attached to the tip of the catheter to expand a stenotic portion of a blood vessel, and a coil for filling an aneurysm in a blood vessel, for example.

In IVR, it is extremely important to continuously and precisely observe the medical device such as a stent and a coil as well as the region of interest. The region of interest refers to an anatomical site serving as a treatment target or an examination target, such as a stenotic or aneurysmal portion of a blood vessel.

Conversely, regions other than the region of interest and/or the medical device do not necessarily need to be continuously and precisely observed. In these regions, it may be preferred to shield or attenuate X-rays from the perspective of reducing X-ray exposure to the object. For the purpose of reducing the X-ray exposure to the object in this manner, the above-described X-ray collimator and/or X-ray filter are provided.

Accordingly, if the aperture opening of the X-ray collimator and the filter opening of the X-ray filter can be precisely matched to the size and position of the region of interest and the medical device, X-ray exposure to the object can be effectively reduced without impeding the procedural efficiency of treatment in IVR. For this purpose, it is first necessary to precisely recognize the region of interest.

### BRIEF DESCRIPTION OF THE DRAWINGS

In the accompanying drawings:

FIG. 1 is a block diagram illustrating a configuration of an X-ray diagnostic apparatus according to the first embodiment;

FIG. 2 is a functional block diagram illustrating a specific configuration of an X-ray irradiator and an X-ray collimator in the X-ray diagnostic apparatus according to the first embodiment, as well as a configuration related to control of the X-ray irradiator;

FIG. 3A is a schematic diagram illustrating a configuration of collimator blades and an aperture opening formed by the collimator blades;

FIG. 3B is a schematic diagram illustrating relationship between the aperture opening and a detection region corresponding to the aperture opening;

FIG. 3C is a schematic diagram illustrating a configuration of an X-ray filter;

FIG. 3D is a schematic diagram illustrating relationship between a filter opening and the detection region corresponding to the filter opening;

FIG. 4 is a flowchart illustrating an operation of the X-ray diagnostic apparatus according to the first embodiment;

FIG. 5A is a schematic diagram for illustrating an operation of the X-ray diagnostic apparatus according to the first embodiment or for illustrating a coil placed within an aneurysm;

FIG. 5B is a schematic diagram for illustrating an operation of the X-ray diagnostic apparatus according to the first embodiment or for illustrating an operation in which the size and position of the filter opening is adjusted;

FIG. 5C is a schematic diagram for illustrating an operation of the X-ray diagnostic apparatus according to the first embodiment or for illustrating an operation in which the position of the filter opening is adjusted in response to coil movement;

FIG. 5D is a schematic diagram for illustrating an operation of the X-ray diagnostic apparatus according to the first embodiment or for illustrating an operation in which the size of the filter opening is adjusted in response to changes in coil size;

FIG. 6 is a flowchart illustrating an operation of the X-ray diagnostic apparatus according to the second embodiment;

FIG. 7A is a schematic diagram for illustrating an operation of the X-ray diagnostic apparatus according to the second embodiment or for illustrating a coil placed within an aneurysm;

FIG. 7B is a schematic diagram for illustrating an operation of the X-ray diagnostic apparatus according to the second embodiment or for illustrating how the size and position of the filter opening is adjusted;

FIG. 7C is a schematic diagram for illustrating an operation of the X-ray diagnostic apparatus according to the second embodiment or for illustrating an operation in which the position of the filter opening is adjusted in response to coil movement;

FIG. 7D is a schematic diagram for illustrating an operation of the X-ray diagnostic apparatus according to the second embodiment or for illustrating an operation in which the size of the filter opening is adjusted in response to changes in size of the coil;

FIG. 8 is a flowchart illustrating an operation of the X-ray diagnostic apparatus according to the third embodiment;

FIG. 9A is a schematic diagram for illustrating an operation of the X-ray diagnostic apparatus according to the third embodiment or for illustrating how the filter opening is set;

FIG. 9B is a schematic diagram for illustrating an operation of the X-ray diagnostic apparatus according to the third embodiment or for illustrating an operation in which the filter opening is adjusted in response to stent movement;

FIG. 9C is a schematic diagram for illustrating an operation of the X-ray diagnostic apparatus according to the third embodiment or for illustrating an operation in which the filter opening is adjusted in response to coil movement.

FIG. 10 is a flowchart illustrating an operation of the X-ray diagnostic apparatus according to the fourth embodiment;

FIG. 11A is a schematic diagram for illustrating an operation of the X-ray diagnostic apparatus according to the fourth embodiment or for illustrating how the filter opening is set;

FIG. 11B is a schematic diagram for illustrating an operation of the X-ray diagnostic apparatus according to the fourth embodiment or for illustrating an operation in which the filter opening is adjusted in response to movement of the tip of a catheter A;

FIG. 11C is a schematic diagram for illustrating an operation of the X-ray diagnostic apparatus according to the fourth embodiment or for illustrating an operation in which the filter opening is adjusted in response to movement of the tip of a catheter B;

FIG. 12 is a flowchart illustrating an operation of the X-ray diagnostic apparatus according to the fifth embodiment;

FIG. 13A is a schematic diagram for illustrating an operation of the X-ray diagnostic apparatus according to the fifth embodiment or for illustrating how the filter opening is set;

FIG. 13B is a schematic diagram for illustrating an operation of the X-ray diagnostic apparatus according to the fifth embodiment or for illustrating an operation in which the filter opening is adjusted in response to movement of the tip of the catheter A; and

FIG. 13C is a schematic diagram for illustrating an operation of the X-ray diagnostic apparatus according to the fifth embodiment or for illustrating an operation in which the filter opening is adjusted in response to movement of the tip of the catheter B.

### DETAILED DESCRIPTION

Hereinbelow, embodiments of the present invention will be described with reference to the accompanying drawings.

In one embodiment, an X-ray diagnostic apparatus includes an X-ray tube, an X-ray collimator, an X-ray detector, and processing circuitry. The X-ray tube generates X-rays. The X-ray collimator forms an opening having higher X-ray transmittance than other regions by using either or both of an X-ray filter configured to attenuate the X-rays and collimator blades configured to shield the X-rays. The X-ray detector detects the X-rays transmitted through an object. The processing circuitry recognizes at least one of a region of interest in the object and a medical device inserted into the object by image recognition on the basis of a detection signal from a detection region of the X-ray detector corresponding to a region of the opening. The processing circuitry outputs a result of the image recognition.

### First Embodiment

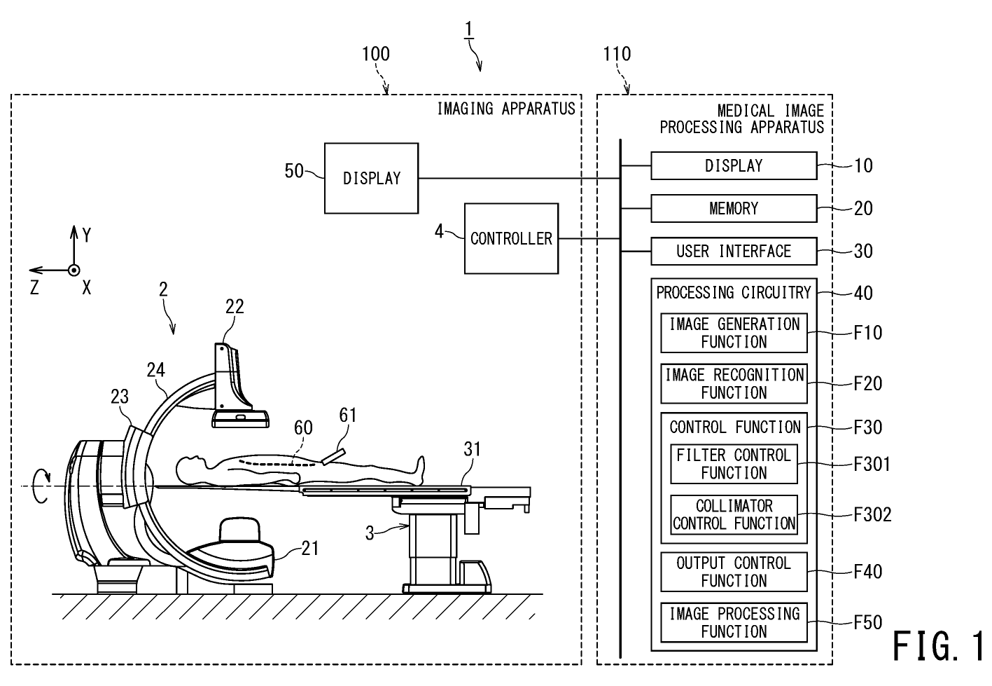

FIG. 1 is a block diagram illustrating a configuration of an X-ray diagnostic apparatus 1 according to the first embodiment. As shown in FIG. 1, the X-ray diagnostic apparatus 1 includes an imaging apparatus 100 and a medical image processing apparatus 110.

As shown in FIG. 1, the imaging apparatus 100 is mainly composed of a gantry 2, a bed 3, and a controller 4. The gantry 2, the bed 3, and the controller 4 are generally installed in a medical procedure room (i.e., an examination/treatment room), whereas the medical image processing apparatus 110 is installed in a control room adjacent to the medical procedure room, for example.

The gantry 2 includes an X-ray irradiator 21, an X-ray detection unit 22, a C-arm driving mechanism 23, and a C-arm 24.

The X-ray irradiator 21 is provided at one end of the C-arm 24. The X-ray irradiator 21 is configured to be movable back and forth under the control of the controller 4. The configuration of the X-ray irradiator 21 will be described in detail below.

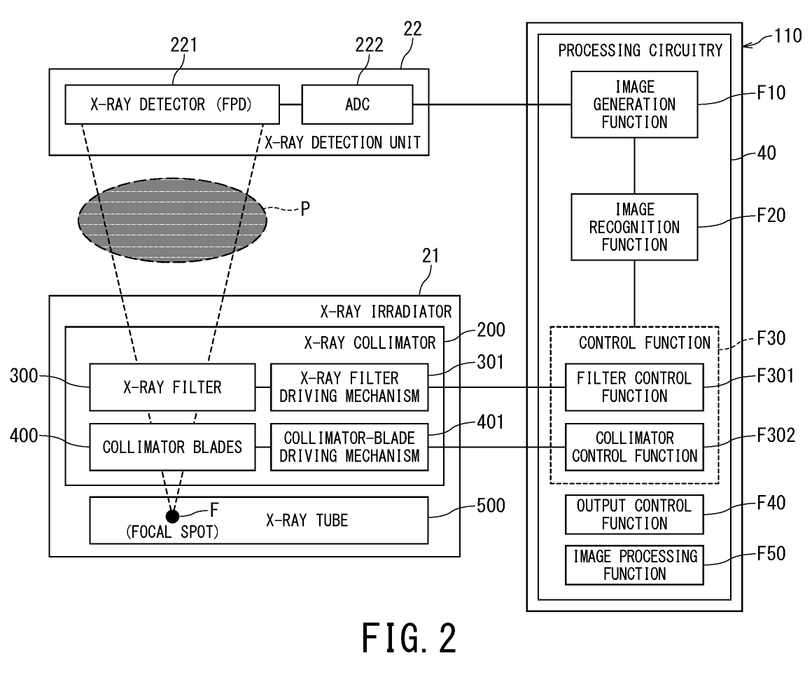

The X-ray detection unit 22 is provided at the other end of the C-arm 24 so as to face the X-ray irradiator 21. The X-ray detection unit 22 is configured to be movable back and forth under the control of the controller 4. For example, the X-ray detection unit 22 includes an X-ray detector 221 constituted by an FPD (Flat Panel Detector) and an ADC (Analog to Digital Converter) 222 (FIG. 2).

The X-ray detector 221 has a plurality of detection elements that are arranged two-dimensionally. Scan lines and signal lines are disposed to intersect each other between the respective detection elements of the X-ray detector 221. A grid may be provided on the front surface of the X-ray detector 221. In order to improve the contrast of X-ray images by absorbing scattered radiation incident on the X-ray detector 221, grid plates formed of a highly X-ray absorptive material such as lead and X-ray transmissive members such as aluminum and wood are arranged alternately in the grid.

The ADC 222 converts projection data of time-series analog signals (video signals) outputted from the X-ray detector 221 into digital signals, and then outputs the digital signals to the medical image processing apparatus 110.

The X-ray detection unit 22 may be an II–TV system (Image Intensifier–Television system). In the II–TV system, X-rays transmitted through the object and X-rays directly incident on the X-ray detector 221 are converted into visible light, and brightness is doubled during conversion from light to electrons and back to light to generate high-sensitivity projection data, and the optical projection data are converted into electrical signals by using a CCD (Charge Coupled Device) imaging sensor.

The C-arm 24 positions both the X-ray irradiator 21 and the X-ray detection unit 22 in such a manner that both face each other with the object interposed at the center of both. Under the control of the controller 4, the C-arm driving mechanism 23 moves the C-arm 24 along the arc direction of the C-arm 24 integrally with both the X-ray irradiator 21 and the X-ray detection unit 22. Although FIG. 1 illustrates a case where the X-ray diagnostic apparatus 1 includes the C-arm 24 configured to move the X-ray irradiator 21 and the X-ray detection unit 22 integrally, the present invention is not limited to such an aspect. For example, the X-ray diagnostic apparatus 1 may be configured such that the X-ray irradiator 21 and the X-ray detection unit 22 are operated independently of each other without including the C-arm 24.

Although FIG. 1 illustrates a configuration of a single-plane X-ray diagnostic apparatus 1 having only one C-arm 24, the X-ray diagnostic apparatus 1 may also be configured as a bi-plane X-ray diagnostic apparatus 1, in which two C-arms enable simultaneous X-ray fluoroscopic imaging from two directions.

The bed 3 is supported on the floor surface and supports a table (i.e., catheter table) 31. Under the control of the controller 4, the bed 3 can slide the table 31 in the X-axis direction and in the Z-axis direction, can move the table 31 up and down in the Y-axis direction, and can roll the table 31. Although FIG. 1 illustrates an under-tube configuration in which the X-ray irradiator 21 is positioned below the table 31, the imaging apparatus 100 may also have an over-tube configuration in which the X-ray irradiator 21 is positioned above the table 31.

The controller 4 includes a CPU (Central Processing Unit) and a memory, which are not shown. Under the control of the medical image processing apparatus 110, the controller 4 controls the driving of the bed 3 and the driving of the X-ray irradiator 21, the X-ray detection unit 22, and the C-arm 24 of the gantry 2. Under the control of the medical image processing apparatus 110, the controller 4 controls the operation of the gantry components such as the X-ray irradiator 21, the X-ray detection unit 22, and the C-arm driving mechanism 23, thereby enabling radiographic or fluoroscopic X-ray imaging for surgical guidance in IVR.

In FIG. 1, a medical device 60 to be used in a medical procedure and a device operation unit 61 are also illustrated. In this specification, the medical device 60 primarily refers to a thin medical instrument that is inserted into a tubular tissue such as a blood vessel for performing diagnosis and/or treatment of the object. Aspects of medical devices to be inserted into blood vessels include: a thin tube called a catheter; a balloon and/or stent attached to the tip of the catheter; a guidewire for guiding the catheter to a diagnostic target site and/or a therapeutic target site within a blood vessel; and a coil for treating an aneurysm, for example.

The device operation unit 61 is an instrument by which a manipulator such as a surgical operator performs manual operations to insert the medical device 60 such as a guidewire and a catheter into a blood vessel and advances the medical device 60 to a predetermined target site.

The medical image processing apparatus 110 is configured based on a computer, such as a workstation and a personal computer. The medical image processing apparatus 110 includes a display 10, a memory 20, a user interface 30, and processing circuitry 40.

The display 10 displays X-ray fluoroscopic images generated by an image generation function F10 of the processing circuitry 40 as well as various support images and support information items, both of which are for assisting a medical procedure such as catheterization and are generated by the processing circuitry 40. The display 50 is a large display device disposed at a position where the display 50 can be easily seen by a manipulator such as a surgical operator during the medical procedure. The display 50 displays X-ray fluoroscopic images (i.e., moving images) and/or X-ray radiographic images (i.e., still images), as well as various support images and support information items. The X-ray fluoroscopic images and X-ray radiographic images are aspects of X-ray images.

The memory 20 is constituted by a hard disk, an optical disk, and a semiconductor memory element such as a RAM (Random Access Memory) and a flash memory, for example. The memory 20 stores various processing programs to be executed by the processing circuitry 40 including application programs and an operating system (OS), as well as data necessary for executing the programs.

The user interface 30 includes: an input device that can be operated by a user; and an input circuit that receives signals from the input device. The input device is constituted by a mouse, a keyboard, a trackball, a switch, a button, a joystick, a touchpad that receives input operations through contact with its operation surface, a touchscreen in which a display and a touchpad are integrated, a non-contact input circuit using an optical sensor, and/or a voice input circuit, for example. When the input device receives an input operation from a user, the input circuit generates an electrical signal corresponding to the input operation and outputs the electrical signal to the processing circuitry 40.

The processing circuitry 40 includes a special-purpose or general-purpose processor and implements various functions described below through software processing by executing the programs stored in the memory 20. The processing circuitry 40 may also be configured to include hardware such as an ASIC (Application Specific Integrated Circuit) and a programmable logic device including an FPGA (Field Programmable Gate Array). The various functions described below can also be implemented through hardware processing by using these devices. Furthermore, the processing circuitry 40 may implement the various functions described below by combining software processing and hardware processing.

The processing circuitry 40 implements: an image generation function F10; an image recognition function F20; a control function F30 that collectively encompasses a filter control function F301 and a collimator control function F302 (i.e., aperture control function F302); an output control function F40; and an image processing function F50.

The image generation function F10 generates X-ray fluoroscopic images as moving images from X-ray detection signals acquired in real time from the X-ray detection unit 22 during a medical procedure using the medical device 60 such as a catheter, and also generates X-ray radiographic images as still images.

On the basis of the X-ray fluoroscopic images generated by the image generation function F10, the image recognition function F20 uses techniques such as machine learning and pattern matching to recognize at least one of the region of interest in the object and the medical device inserted in the object by image recognition.

Each of the filter control function F301 and the collimator control function F302 in the control function F30 controls the aperture of the X-ray collimator 200 (FIG. 2) in the X-ray irradiator 21 on the basis of either or both of the region of interest of the object and the medical device inserted in the object, both of which are recognized by image recognition.

The output control function F40 outputs various data to the display 10 as an output unit. Consequently, the output control function F40 causes the display 10 to display various data.

On the basis of the result of the image recognition, the image processing function F50 performs image processing on the region including at least one of the recognized region of interest and the recognized medical device. This image processing includes segmentation processing, image processing with a processing time exceeding a threshold, and image processing with a number of processing steps exceeding a threshold, for example. This image processing may also involve transferring image data to an image-processing server connected via a network and receiving a processing result from this image-processing server. Further, the image processing may be AI (Artificial Intelligence) processing, and may be processing for determining how a coil is positioned in an aneurysm sac, for example.

The operations of the image recognition function F20 and the control function F30 will be described in detail below. Before describing the detailed operations of the image recognition function F20 and the control function F30 in the X-ray diagnostic apparatus 1 according to the first embodiment, a description will be given of: (i) the configuration of the X-ray collimator 200 provided in the X-ray diagnostic apparatus 1; and (ii) the relationship between the opening (i.e., the region through which X-rays pass or the region having a higher X-ray transmittance than other regions) in the X-ray collimator 200 and the detection region of the X-ray detector 221.

FIG. 2 is a functional block diagram illustrating a specific configuration of the X-ray irradiator 21 and the X-ray collimator 22 in the X-ray diagnostic apparatus 1 according to the first embodiment, as well as a configuration related to the control of the X-ray irradiator 21.

As shown in FIG. 2, the X-ray irradiator 21 includes the X-ray collimator 200 and an X-ray tube 500. Of these components, the X-ray tube 500 generates X-rays by receiving a high voltage supply from a high-voltage power source (not shown). The X-rays generated by the X-ray tube 500 pass from the X-ray focal spot F through the opening(s) of either or both of the X-ray filter 300 and the collimator blades 400 of the X-ray collimator 200, then pass through the object P, and are detected by the X-ray detector 221.

As described above, the X-ray detector 221 is configured as an FPD, for example. The analog detection signals detected by the respective detection elements of the FPD are converted into digital detection signals by the ADC 222. On the basis of these detection signals, X-ray fluoroscopic images or X-ray radiographic images are generated by the image generation function F10 of the processing circuitry 40 in the medical image processing apparatus 110.

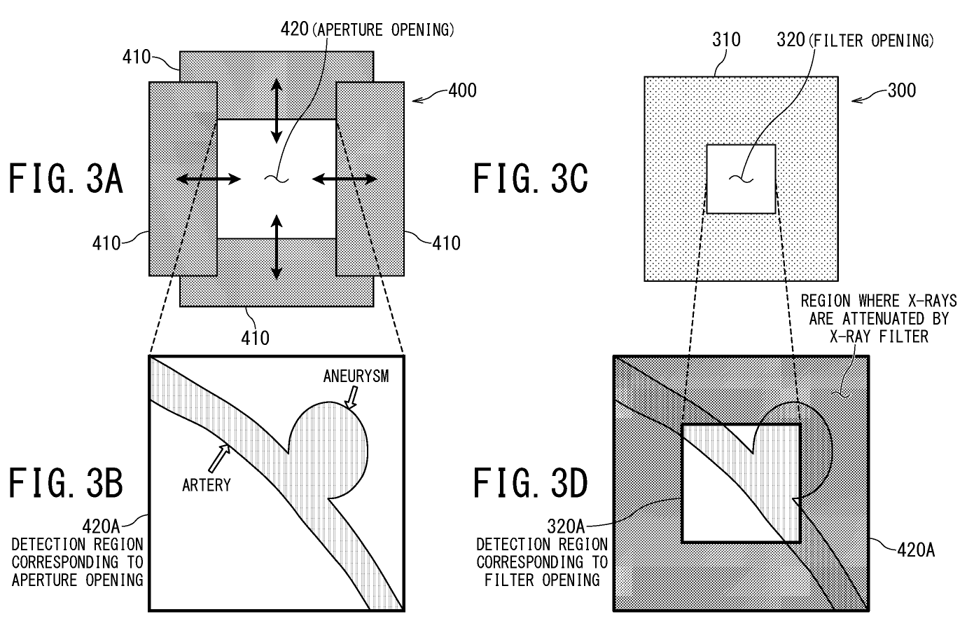

FIG. 3A is a schematic diagram illustrating a configuration of the collimator blades 400 included in the X-ray collimator 200 and the aperture opening 420 formed by the collimator blades 400. The collimator blades 400 are plate members configured to shield X-rays and are formed of, for example, lead plates. The collimator blades 400 are composed of, for example, four shielding blades 410, each of which is independently driven in the open and closed directions as indicated by the black arrows in FIG. 3A. The four shielding blades 410 are independently driven and thereby allow the size, shape (i.e., aspect ratio of the rectangle), and position of the aperture opening 420 to be adjusted as desired.

Under the control of the collimator control function F302 as shown in FIG. 2, each of the shielding blades 410 of the aperture opening 420 is independently driven by the collimator-blade driving mechanism 401 in such a manner that the size, shape, and position of the aperture opening 420 can be set or adjusted as desired.

Of all the regions of the X-ray detector 221, the detection region 410A corresponding to the aperture opening 420 detects the X-rays, which have passed through the aperture opening 420 and then through the object P, as shown in FIG. 3B. In the detection region 410A, at least one of the region of interest in the object and the medical device is depicted.

As described above, the region of interest refers to an anatomical site that is a treatment target and/or an examination target, such as a stenotic site of a blood vessel and a site of an aneurysm. In addition, the medical device refers to a device to be used for treating or examining a diseased site of a blood vessel, and aspects of the medical device include: a thin tube called a catheter; a balloon and/or stent attached to the tip of the catheter; a guidewire for guiding the catheter to a diagnostic target site or therapeutic target site within the blood vessel; and a coil for treating an aneurysm, for example.

FIG. 3C schematically illustrates a configuration of the X-ray filter 300 provided in the X-ray collimator 200 and the filter opening 320 formed by the X-ray filter 300. The X-ray filter 300 is a plate member for attenuating and transmitting X-rays and is formed of, for example, a copper plate or an aluminum plate. The X-ray filter 300 is composed of, for example, a single filter plate 310, which is driven in a direction perpendicular to the sheet of FIG. 3C (i.e., in the depth direction). Driving the single filter plate 310 allows the position of the filter opening 320 to be arbitrarily adjusted as desired.

Under the control of the filter control function F301 as shown in FIG. 2, the filter plate 310 of the X-ray filter 300 is driven by the X-ray filter driving mechanism 301 in such a manner that the position of the filter opening 320 can be set or adjusted.

The X-ray collimator 200 can be configured with only the collimator blades 400 without providing the X-ray filter 300. Additionally or alternatively, as shown in FIG. 2, the X-ray collimator 200 can be configured to include the X-ray filter 300 in addition to the collimator blades 400. When the X-ray collimator 200 is provided with both the collimator blades 400 and the X-ray filter 300, the size and position of the filter opening 320 are typically controlled in such a manner that the detection region 320A corresponding to the filter opening 320 is disposed inside the detection region 420A corresponding to the aperture opening 420 as shown in FIG. 3D.

As described above, the X-ray filter 300 is also referred to as an ROI (Region of Interest) filter. The X-ray filter 300 attenuates X-rays in regions other than the region of interest (ROI), while allowing X-rays to pass through the filter opening 320 without attenuation. In IVR, it is extremely important to continuously and precisely observe the region of interest (i.e., the anatomical site that is the target of treatment or examination, such as a stenotic site and an aneurysmal site in a blood vessel) and/or a medical device, such as a stent and a coil. For this reason, the filter opening 320 needs to allow X-rays to pass through a relatively narrow region containing the region of interest and the medical device without attenuation. Conversely, for regions other than the region of interest and the medical device, it may be preferred to shield or attenuate X-rays to reduce X-ray exposure to the object.

Although regions other than the region of interest and the medical device do not necessarily need to be observed continuously and precisely, there is also a demand for obtaining information on a wider region outside the region of interest and the medical device to some extent during a medical procedure. From this perspective, the X-ray collimator 200 is provided with the X-ray filter 300 in addition to the collimator blades 400.

Due to the effect of the X-ray filter 300, clear and highly visible X-ray fluoroscopic images are generated in the detection region 320A corresponding to the filter opening 320 as shown in FIG. 3D, whereas X-rays in regions outside the filter opening 320 are attenuated to suppress X-ray exposure to the object and exhibit reduced visibility to some extent.

In the X-ray diagnostic apparatus 1 of each embodiment described below, at least one of the region of interest and the medical device is recognized by image recognition on the basis of the detection signals detected in the detection region 320A corresponding to the filter opening 320, and the filter opening 320 is controlled on the basis of at least one of the recognized region of interest and the recognized medical device.

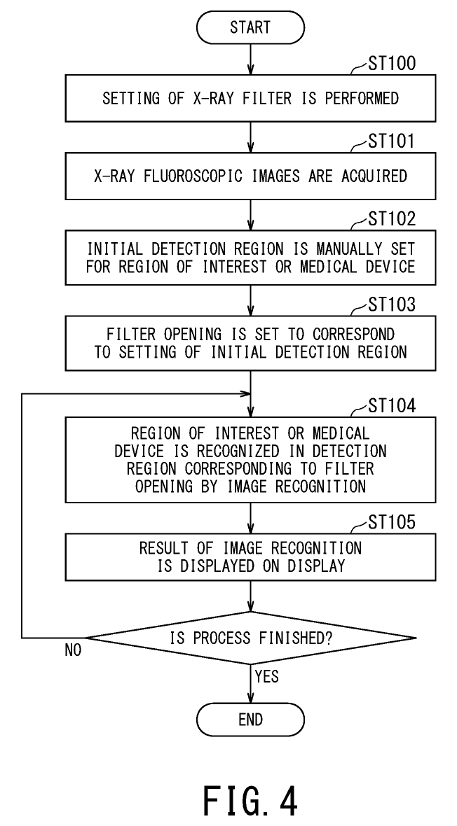

Hereinafter, the operation of each embodiment will be described in detail with reference to the flowcharts and operation diagrams shown in FIGS. 4 to 13C.

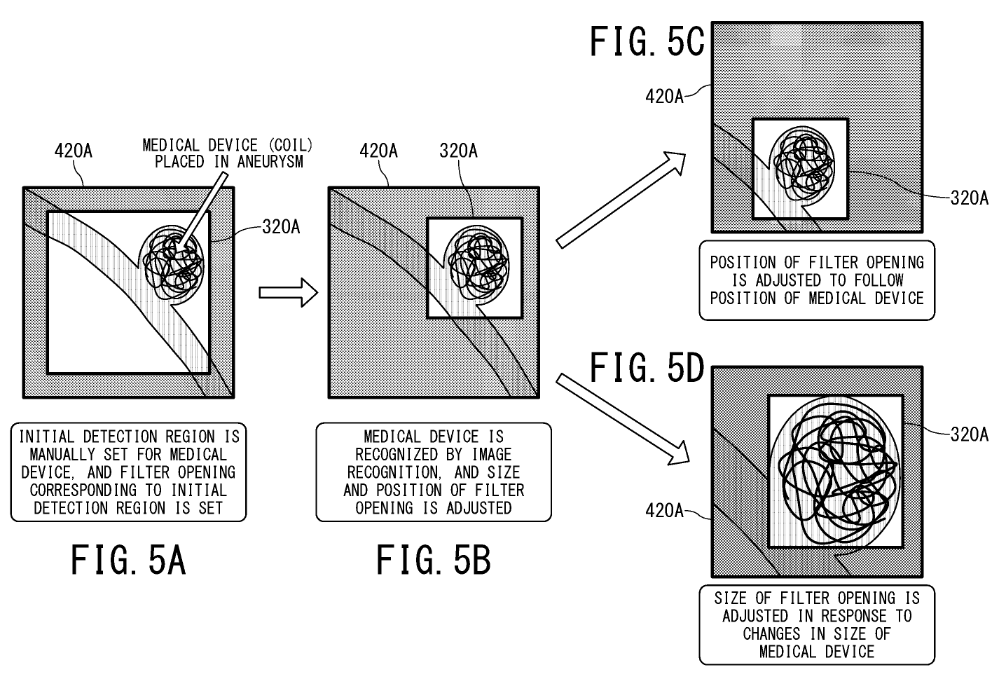

FIG. 4 is a flowchart illustrating an operation of the X-ray diagnostic apparatus 1 according to the first embodiment. FIGS. 5A to 5D are schematic diagrams for illustrating an operation of the X-ray diagnostic apparatus 1 according to the first embodiment.

First, in Step ST100, setting of the X-ray filter 300 is performed. This setting of the X-ray filter 300 means setting both the collimator blades 400 and the X-ray filter 300 in the X-ray collimator 200, in which the collimator blades 400 are always set but the X-ray filter 300 can be set as an option.

In subsequent Step ST101, the object is irradiated with X-rays and X-ray fluoroscopic images are acquired.

In Step ST102, while observing the X-ray fluoroscopic images, a user such as a doctor manually sets an initial detection region 320A via the user interface 30 for the region of interest or for the medical device depicted in the X-ray fluoroscopic images. Thereafter, the processing circuitry 40 continues searching the initial detection region 320A for the region of interest.

In Step ST103, in response to this manual setting, the filter opening 320 is set to correspond to the initial detection region 320A. The setting of the filter opening 320 is performed by the X-ray filter driving mechanism 301 driving the filter plate 310 under the control of the filter control function F301 of the processing circuitry 40.

FIG. 5A is an operation diagram corresponding to Step ST102 and Step ST103. In FIG. 5A, a coil placed in or deployed into an aneurysm is shown as one aspect of the medical device, and the user manually sets the initial detection region 320A in such a manner that this coil is included in the initial detection region 320A.

In subsequent Step ST104, the image recognition function F20 of the processing circuitry 40 performs image recognition processing based on pattern matching and/or machine learning on the X-ray fluoroscopic images in the initial detection region 320A to recognize the region of interest or the medical device by image recognition. In other words, the image recognition function F20 calculates a detection region on the basis of the user operation on either or both of the collimator blades 400 and the X-ray filter 300, and recognizes at least one of the region of interest and the medical device in the detection region in the X-ray fluoroscopic images acquired in Step ST101. The image recognition function F20 may recognize both the aneurysm and the stent as the region of interest and the medical device, for example.

In Step ST105, the output control function F40 causes the display 10 to display the result of the image recognition. Thereafter, the X-ray diagnostic apparatus 1 may transition to a first mode in which the filter control function F301 adjusts the size and/or position of the filter opening 320 in response to changes in the size and/or position of the region of interest or the medical device on the basis of the result of the image recognition. Additionally or alternatively, the X-ray diagnostic apparatus 1 may transition to a second mode in which the image processing function F50 performs image processing on a region including at least one of the recognized region of interest and the recognized medical device on the basis of the result of the image recognition.

The processing circuitry 40 may execute both the first mode and the second mode in parallel or may execute only one of both.

FIGS. 5B to 5D are operation diagrams corresponding to the processing of Step ST104 and Step ST105. FIG. 5B illustrates how the size and position of the filter opening 320 are adjusted by using the result of the image recognition so as to include the entire coil and minimize the inclusion of regions other than the coil despite unchanged size and/or position of the coil of the medical device. Such adjustment based on the image recognition enables acquisition of clear and highly visible X-ray fluoroscopic images in the region including the coil, while reducing radiation exposure in regions of the object other than the coil. As a result, this configuration can provide a user such as a doctor with a satisfactory treatment environment while also enabling highly reliable image recognition processing.

FIG. 5C illustrates an operation case in which the position of the filter opening 320 is adjusted to follow the movement of the coil even if the angle and/or position of the C-arm 24 and/or table 31 changes depending on the treatment situation and/or diagnostic situation and the position of the coil within the aperture opening 420 changes accordingly.

FIG. 5D illustrates an operation case in which the size of the filter opening 320 is adjusted in response to changes in size of the coil even if the size of the coil depicted in the X-ray fluoroscopic images changes by changing the SID (Source to Image Distance) or the magnification of the X-ray detector 221 depending on the treatment situation or the diagnostic situation.

According to the operation of the first embodiment described above, even if the position and/or size of the medical device such as a coil changes depending on the treatment situation or diagnostic situation, the X-ray diagnostic apparatus 1 can acquire clear and highly visible X-ray fluoroscopic images in the region including the coil while reducing radiation exposure to regions of the object other than the coil as the medical device. As a result, the X-ray diagnostic apparatus 1 can provide a user such as a doctor with a satisfactory treatment environment while simultaneously enabling highly reliable image recognition processing.

Although a description has been given of the case where the medical device such as a coil is recognized by image recognition and thereby the filter opening 320 is adjusted so as to follow changes in the size and/or position of the medical device, the tracking target is not limited to the medical device. For example, a specific branching point of a blood vessel after administration of a contrast medium and/or the regions of interest such as an arterial aneurysm and a venous aneurysm can also be recognized by image-recognition, and the size and/or position of the filter opening 320 can be adjusted so as to follow changes in the relative position and/or size of these regions in the X-ray fluoroscopic images.

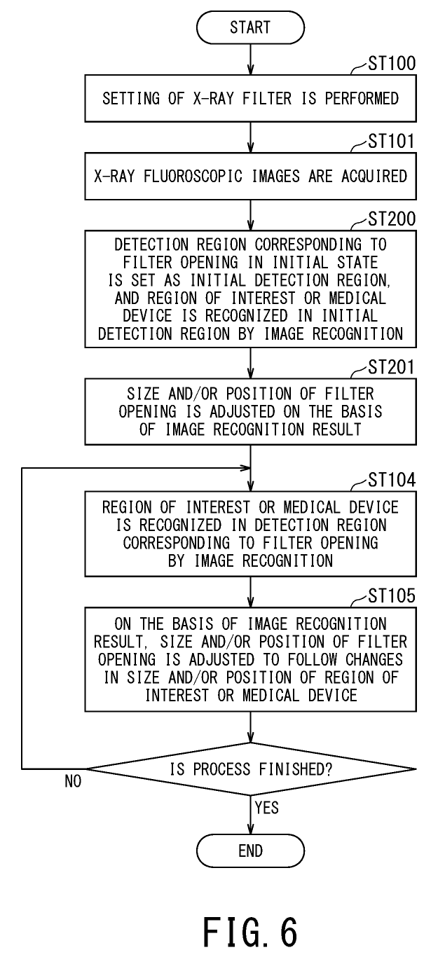

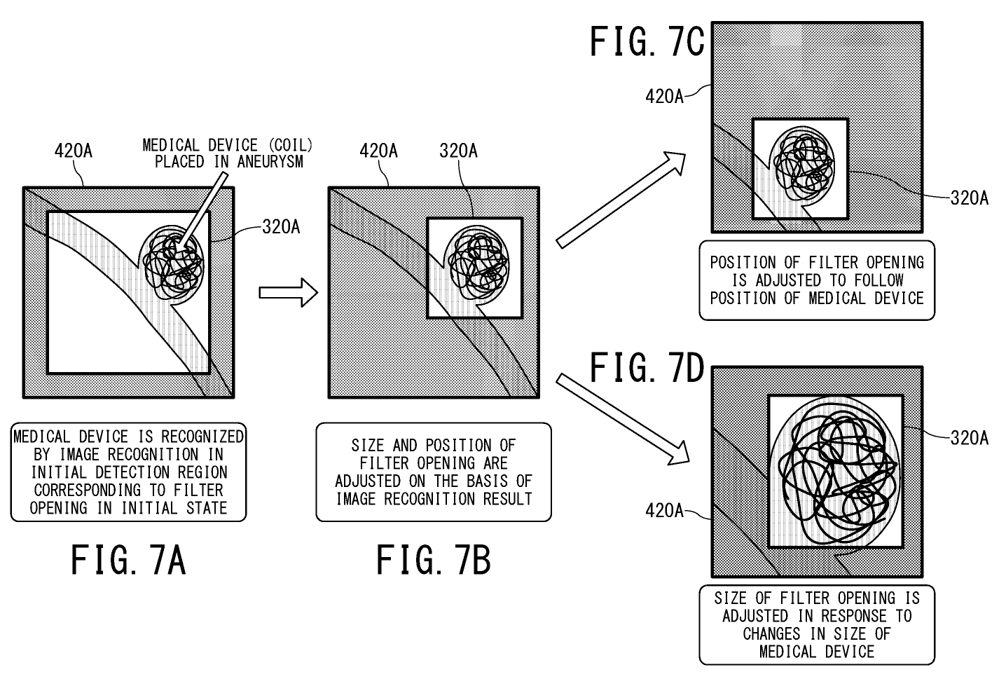

FIG. 6 is a flowchart illustrating an operation of the X-ray diagnostic apparatus 1 according to the second embodiment. FIG. 7A to FIG. 7D are schematic diagrams for illustrating an operation of the X-ray diagnostic apparatus 1 according to the second embodiment.

In FIG. 6, the difference from the first embodiment lies in the processing of Step ST200 and Step ST201.

In Step ST200, the detection region corresponding to the initial filter opening 320 is set as the initial detection region 320A, and the region of interest or the medical device is recognized by image recognition in the initial detection region 320A. FIG. 7A is an operation diagram corresponding to the processing of Step ST200.

In Step ST201, on the basis of the result of the image recognition, the size and/or position of the filter opening 320 is adjusted. FIG. 7B is an operation diagram corresponding to the processing of Step ST201.

Although it is assumed in the second embodiment that the region of interest or the medical device (i.e., the coil as the medical device in the case of FIGS. 7A to 7D) exists within the initial detection region 320A, the second embodiment eliminates the need for the user to manually set the initial detection region, as in Step ST102 and Step ST103 of the first embodiment.

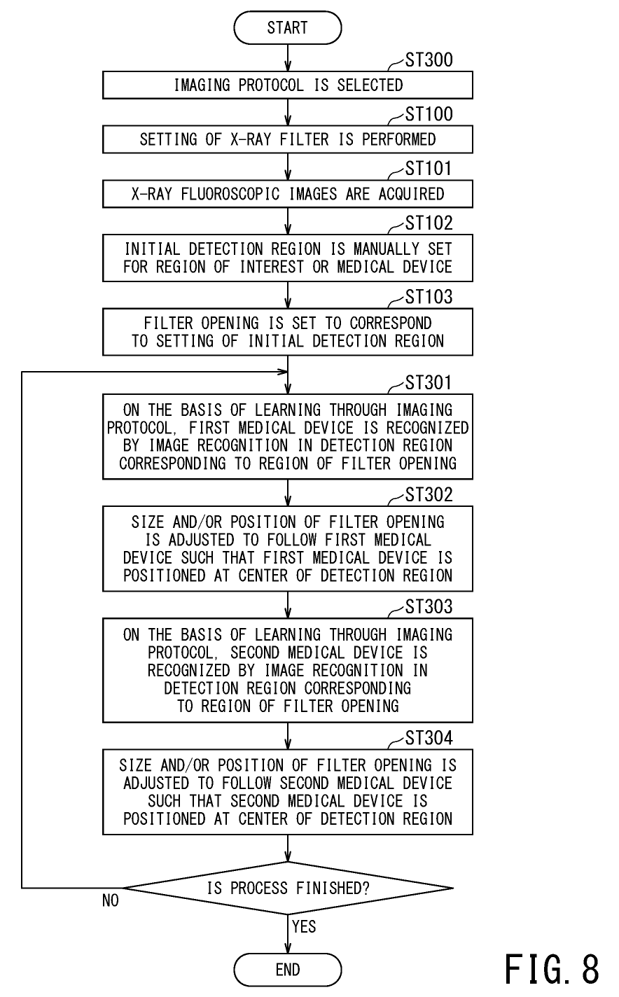

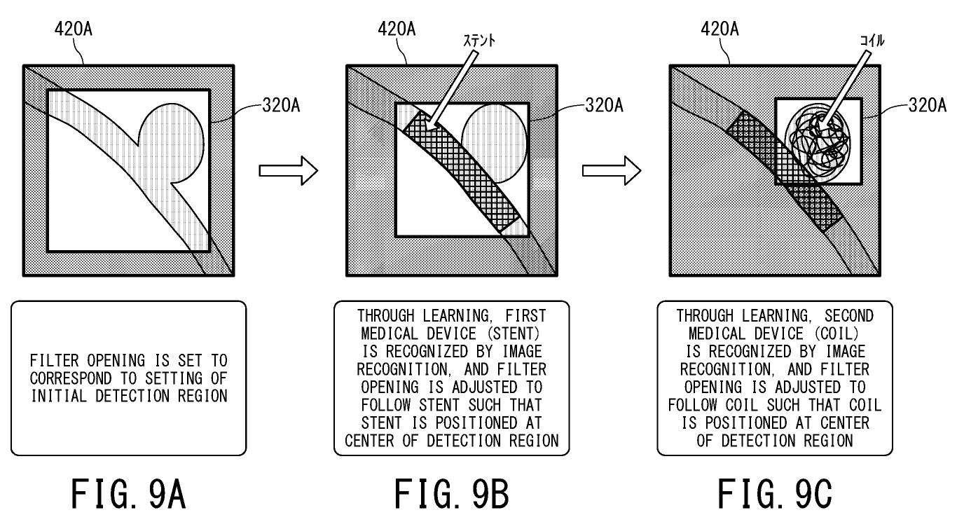

FIG. 8 is a flowchart illustrating an operation of the X-ray diagnostic apparatus 1 according to the third embodiment. FIG. 9A to FIG. 9C are schematic diagrams for illustrating an operation of the X-ray diagnostic apparatus 1 according to the third embodiment.

In the third embodiment, the processing circuitry 40 learns in advance that a plurality of different medical devices enter the detection region in a predetermined order, and controls the position of the opening on the basis of the learning results (i.e., trained result) in such a manner that each of the medical devices sequentially entering the detection region is positioned at the center of the detection region.

For example, in a treatment method involving placement of a coil in an aneurysm, depending on the size and shape of the aneurysm neck, the coil may not remain stable within the aneurysm. In order to address this problem, there is a treatment known as stent-assisted coil embolization, in which a stent is first placed at the aneurysm neck and a coil is then placed into the aneurysm.

In Step ST300 in FIG. 8, a user selects an imaging protocol corresponding to such a treatment method via the user interface 30.

The processing from Step ST100 to Step ST103 is the same as in the first embodiment, and duplicate description is omitted. FIG. 9A is an operation diagram for illustrating processing corresponding to Step ST100 to Step ST103.

In Step ST301, the processing circuitry 40 recognizes the first medical device (i.e., a stent in this case) by image recognition in the detection region 320A corresponding to the region of the filter opening 320 through learning based on the imaging protocol.

In subsequent Step ST302, the size and/or position of the filter opening 320 is adjusted to track and match the stent (i.e., the first medical device) in such a manner that the stent is positioned at the center of the detection region 320A.

FIG. 9B is a schematic diagram illustrating processing corresponding to Step ST301 and Step ST302.

In Step ST303, the processing circuitry 40 continues to recognize a second medical device (i.e., a coil in this case) by image recognition in the detection region 320A corresponding to the region of the filter opening 320 through learning based on the imaging protocol.

In Step ST304, the size and/or position of the filter opening 320 is adjusted to track and match the coil (i.e., the second medical device) in such a manner that: (i) the coil is positioned at the center in the detection region 320A; and (ii) regions outside the coil are not significantly included within the detection region 320A.

FIG. 9C is an operation diagram illustrating processing corresponding to Step ST301 and Step ST302.

According to the third embodiment, even in a medical procedure in which a plurality of medical devices are sequentially inserted into a region near the region of interest (e.g., an aneurysm), the size and/or position of the filter opening 320 can be adjusted so as to automatically follow the coil and match the position and size of the coil on the basis of the imaging protocol and machine learning in such a manner that the plurality of medical devices are sequentially positioned at the center of the filter opening 320 without any user intervention.

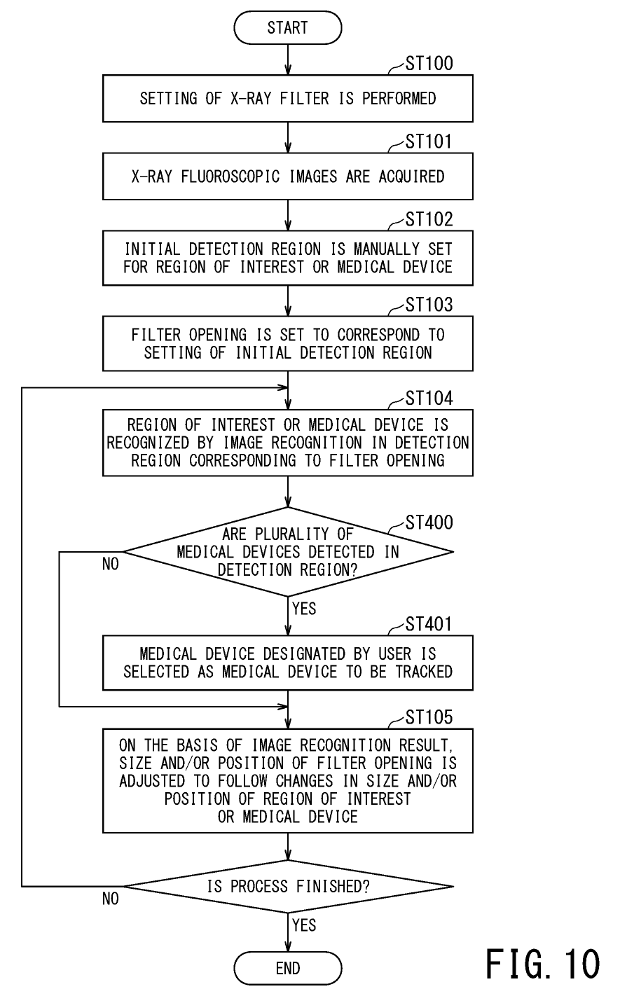

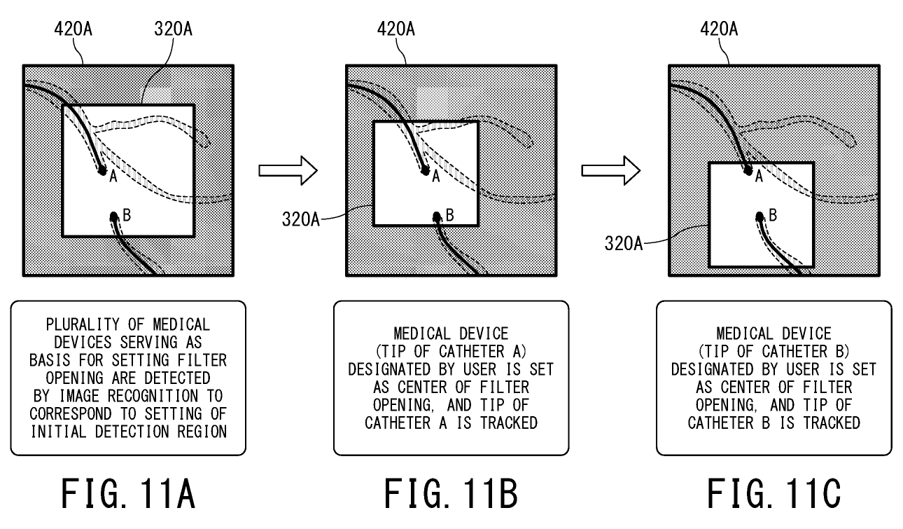

FIG. 10 is a flowchart illustrating an operation of the X-ray diagnostic apparatus 1 according to the fourth embodiment. FIGS. 11A to 11C are schematic diagrams for illustrating an operation of the X-ray diagnostic apparatus 1 according to the fourth embodiment.

For example, in the case of a patient with a lesion known as chronic total occlusion (CTO), a medical procedure in which the stenosis is approached from a plurality of directions using catheters is reported to be effective.

In this case, the X-ray fluoroscopic images of the stenosis depict the tips of the respective catheters (for example, two catheters) approaching the stenosis from different directions. Typically, the surgical operator, who is the user, does not operate both catheters simultaneously, and thus focuses on the position and movement of the tip of one of the catheters.

Accordingly, in the fourth embodiment, when a plurality of medical devices (e.g., tips of respective catheters) are detected in the detection region 320A, the position of the filter opening 320 is controlled in such a manner that one medical device selected or designated by the user from among these depicted medical devices is positioned at the center of the detection region 320A.

The processing from Step ST100 to Step ST103 is the same as in the first embodiment, and duplicate description is omitted. FIG. 11A is an operation diagram illustrating processing corresponding to Step ST100 to Step ST104.

In Step ST400, it is determined by image recognition whether a plurality of medical devices are present within the detection region 320A or not. If a plurality of medical devices are present in the detection region 320A, the routine proceeds to Step ST401.

In Step ST401, the medical device designated by the user is selected as the medical device to be tracked.

In subsequent Step ST105, on the basis of the result of the image recognition for the medical device designated by the user, the size and/or position of the filter opening 320 is adjusted in response to changes in the size and/or position of the region of interest or the medical device.

FIGS. 11A and 11B illustrates a case where two medical devices including the tip of a catheter A and the tip of a catheter B exist within the detection region 320A and the user designates the catheter A so that the tip of the catheter A is set at the center of the filter opening 320 and is tracked.

Step ST104 and Step ST105 are repeated until the process is completed. If the medical device designated by the user is changed from the tip of the catheter A to the tip of the catheter B in Step ST401, after this change of designation, the tip of the catheter B is then set at the center of the filter opening 320 and is followed or tracked as shown in FIG. 11C.

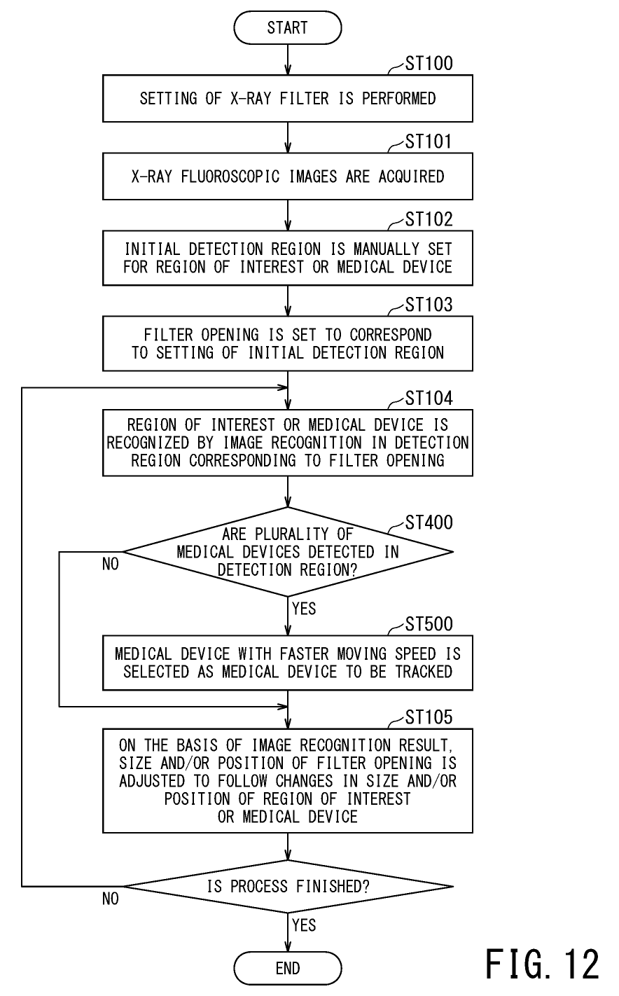

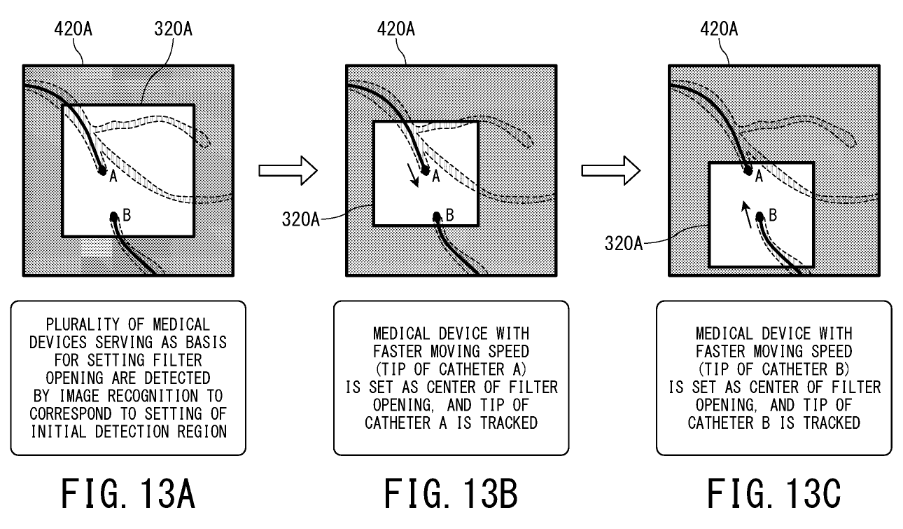

FIG. 12 is a flowchart illustrating an operation of the X-ray diagnostic apparatus 1 according to the fifth embodiment. FIGS. 13A to 13C are schematic diagrams for illustrating an operation of the X-ray diagnostic apparatus 1 according to the fifth embodiment. The difference between the flowchart of FIG. 12 for the fifth embodiment and the flowchart of FIG. 10 for the fourth embodiment lies only in the processing of Step ST500 and Step ST401.

In the fourth embodiment, when a plurality of medical devices exist within the detection region 320A, the selection of which medical device to set at the center of the filter opening 320 and to track is determined depending on a designation by a user.

Contrastively, in the fifth embodiment, when a plurality of medical devices exist within the detection region 320A, of these medical devices each having its own moving speed, the medical device with the fastest moving speed is automatically set at the center of the filter opening 320 and is automatically tracked.

In Step ST500, for example, when the respective tips of the catheters A and B are present within the detection region 320A as shown in FIGS. 13A and 13B, the medical device with the faster moving speed (the tip of the catheter A in the case of FIG. 13B) is selected as the tracking-target medical device.

In subsequent Step ST105, the tip of the selected catheter A is set at the center of the filter opening 320 and the tip of catheter A is tracked.

When the user is operating the catheter A of the two catheters A and B, it is considered that the moving speed of the tip of the catheter A is faster than the moving speed of the tip of the catheter B. In this case, the tip of the catheter A being operated by the user is automatically selected as the tracking target, thereby reducing the operational burden on the user as compared with the fourth embodiment.

The processing from Step ST104 to Step ST105 is repeated until the process is completed. If the user's operation target shifts from the catheter A to the catheter B, it is likely that the moving speed of the tip of the catheter B becomes faster than the moving speed of the tip of the catheter A. In this case, as shown in FIG. 13C, the tracking target is automatically switched to the tip of the catheter B, which is currently being operated by the user, and thus, the operational burden on the user is reduced as compared with the fourth embodiment.

So far, descriptions have been given of the embodiments in which the size and/or position of the filter opening 320 of the X-ray filter (i.e., ROI filter) 300 are adjusted to follow changes in the size and/or position of the region of interest and/or the medical device. However, embodiments of the present invention are not limited to the above-described aspect, and the size and/or position of the aperture opening 420 of the collimator blades 400 may be adjusted to follow changes in the size and/or position of the region of interest and/or the medical device. In this case, the filter opening 320 described in each of the above-described embodiments may be read by replacing it with the aperture opening 420, and the same technical effects as in each of the above-described embodiments can be obtained.

In the sixth embodiment, a description will be given of how to expand the search range when any medical device cannot be detected in the X-ray images in the case of changing the angle of the C-arm 24.

If the medical device is not detected in the region of interest in the X-ray fluoroscopic images, it is assumes that the medical device is outside the region of interest and the processing circuitry 40 searches around the region of interest for the medical device. In order to achieve this, the search range is expanded on the basis of the information on the C-arm 24 and the location of the bed 3.

For example, when the image recognition function F20 has previously recognized a medical device in the X-ray fluoroscopic images at the previous position of the C-arm 24, the processing circuitry 40 cause the memory 20 to store the pre-acquired information (i.e., prior information) including the previous position of the C-arm 24. When the image recognition function F20 cannot recognize the medical device in the X-ray fluoroscopic images, the processing circuitry 40 starts searching for the medical device from the position of the C-arm 24 contained in the pre-acquired information in the memory 20 and expands the search range. In other words, the processing circuitry 40 searches the vicinity of the region of interest identified by the pre-acquired information, i.e., performs a search over a range slightly larger than the region of interest.

For example, because a lesion such as a cerebral aneurysm is small, the processing circuitry 40 first expands the search range to a normal field of view (i.e., wide field of view) and then searches again.

If the medical device is still not detected, the processing circuitry 40 may extend the SID (X-ray Source to Image Receptor Distance). SID is the distance between the focal spot F of the X-ray tube 500 and the surface of the X-ray detector 221, i.e., the imaging distance. If the medical device is still not detected even after extending SID, the output control function F40 may cause the display 10 to display a message indicating that the medical device is not detected.

As described above, the X-ray diagnostic apparatus 1 of each embodiment can precisely recognize a region of interest in the object and/or the region including the medical device.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
an X-ray tube configured to generate X-rays;
an X-ray collimator configured to form an opening having higher X-ray transmittance than other regions by using either or both of an X-ray filter for attenuating the X-rays and collimator blades for shielding the X-rays;
an X-ray detector configured to detect the X-rays transmitted through an object; and
processing circuitry configured to
recognize at least one of a region of interest in the object and a medical device inserted into the object by image recognition based on a detection signal from a detection region of the X-ray detector corresponding to a region of the opening and
output a result of the image recognition.

2. The X-ray diagnostic apparatus according to claim 1, wherein the processing circuitry is configured to:
calculate the detection region depending on a user operation on either or both of the X-ray filter and the collimator blades; and
recognize at least one of the region of interest and the medical device in the detection region in an acquired X-ray image.

3. The X-ray diagnostic apparatus according to claim 2, wherein the processing circuitry is configured to perform image processing on a region including at least one of a recognized region of interest and a recognized medical device by using the result of the image recognition.

4. The X-ray diagnostic apparatus according to claim 1, wherein:
the processing circuitry is configured to control the opening of the X-ray collimator depending on at least one of a recognized region of interest and a recognized medical device; and
the X-ray collimator is configured to adjust a size or position of the opening by driving either or both of the X-ray filter and the collimator blades in accordance with a control signal from the processing circuitry.

5. The X-ray diagnostic apparatus according to claim 4, wherein the processing circuitry is configured to control the opening in such a manner that a size or position of the detection region matches a size or position of at least one of the region of interest and the medical device.

6. The X-ray diagnostic apparatus according to claim 4, wherein the processing circuitry is configured to:
recognize movement of at least one of the region of interest and the medical device; and
control a position of the opening in such a manner that the detection region is adjusted to follow the region of interest and the medical device when a position of at least one of the region of interest and the medical device changes.

7. The X-ray diagnostic apparatus according to claim 5, wherein the processing circuitry is configured to:
recognize movement of at least one of the region of interest and the medical device; and
control a position of the opening in such a manner that the detection region is adjusted to follow the region of interest and the medical device when a position of at least one of the region of interest and the medical device changes.

8. The X-ray diagnostic apparatus according to claim 4, wherein, when a plurality of medical devices are detected in the detection region, the processing circuitry controls the position of the opening in such a manner that a medical device designated by a user among the plurality of medical devices is positioned at a center of the detection region.

9. The X-ray diagnostic apparatus according to claim 4, wherein the processing circuitry is configured to:
learn in advance that a plurality of different medical devices enter the detection region in a predetermined order; and
control a position of the opening depending on a learning result in such a manner that each of the plurality of medical devices sequentially entering the detection region is positioned at a center of the detection region.

10. The X-ray diagnostic apparatus according to claim 4, wherein, when a plurality of medical devices with different moving speeds are detected in the detection region, the processing circuitry controls a position of the opening in such a manner that the medical device with a faster moving speed is positioned at a center of the detection region.

11. A medical image processing apparatus comprising processing circuitry configured to:
acquire a detection signal from a detection region of an X-ray detector by using either or both of an X-ray filter for attenuating X-rays and collimator blades for shielding the X-rays, the detection region corresponding to a region of an opening having higher X-ray transmittance than other regions;
recognize at least one of a region of interest of an object and a medical device inserted into the object by image recognition based on the detection signal; and
output a result of the image recognition.

12. The medical image processing apparatus according to claim 11, wherein the processing circuitry is configured to:
calculate the detection region depending on a user operation on either or both of the X-ray filter and the collimator blades; and
recognize at least one of the region of interest and the medical device in the detection region in an acquired X-ray image.

13. The medical image processing apparatus according to claim 12, wherein the processing circuitry is configured to perform image processing on a region including at least one of a recognized region of interest and a recognized medical device by using the result of the image recognition.

14. The X-ray diagnostic apparatus according to claim 11, wherein the processing circuitry is configured to control an opening of a X-ray collimator depending on at least one of a recognized region of interest and a recognized medical device, the X-ray collimator being configured to form the opening having higher X-ray transmittance than other regions by using either or both of the X-ray filter and the collimator blades.

15. The X-ray diagnostic apparatus according to claim 14, wherein the processing circuitry is configured to control the opening in such a manner that a size or position of the detection region matches a size or position of at least one of the region of interest and the medical device.

16. A control method for an X-ray diagnostic apparatus comprising steps of:
acquiring a detection signal from a detection region of an X-ray detector by using either or both of an X-ray filter for attenuating X-rays and collimator blades for shielding the X-rays, the detection region corresponding to a region of an opening having higher X-ray transmittance than other regions;
recognizing at least one of a region of interest of an object and a medical device inserted into the object by image recognition based on the detection signal; and
outputting a result of the image recognition.

17. The control method for an X-ray diagnostic apparatus according to claim 16 further comprising steps of:
calculating the detection region depending on a user operation on either or both of the X-ray filter and the collimator blades; and
recognizing at least one of the region of interest and the medical device in the detection region in an acquired X-ray image.

18. The control method for an X-ray diagnostic apparatus according to claim 17 further comprising a step of performing image processing on a region including at least one of a recognized region of interest and a recognized medical device by using the result of the image recognition.

19. The control method for an X-ray diagnostic apparatus according to claim 16 further comprising a step of controlling an opening of a X-ray collimator depending on at least one of a recognized region of interest and a recognized medical device, the X-ray collimator being configured to form the opening having higher X-ray transmittance than other regions by using either or both of the X-ray filter and the collimator blades.

20. The control method for an X-ray diagnostic apparatus according to claim 19 further comprising a step of controlling the opening in such a manner that a size or position of the detection region matches a size or position of at least one of the region of interest and the medical device.
